(19) United States
(12) Patent Application Publication
(10) Pub. No.: US 2009/0074671 A1
(43) Pub. Date: Mar. 19, 2009

(54) DRUG-IMMOBILIZED INORGANIC NANOPARTICLE

(75) Inventors: Masayoshi Kojima, Kanagawa (JP); Hiroyuki Hirai, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Minato-ku, TOKYO (JP)

(21) Appl. No.: 12/293,331

(22) PCT Filed: Mar. 30, 2007
(86) PCT No.: PCT/JP2007/057709

§ 371 Date: Sep. 17, 2008

(30) Foreign Application Priority Data
Mar. 30, 2006 (JP) 2006-093451

(51) Int. Cl. A61K 9/14; A61K 49/18

(52) U.S. Cl. 424/9.3; 424/489

(57) ABSTRACT

An object of the present invention is to provide a composite of an inorganic nanoparticle with a medicinal compound (i.e., an active substance), and to provide a method for producing the inorganic nanoparticle composite, which is highly versatile and convenient. The present invention provides an active substance-immobilized inorganic nanoparticle wherein an active substance is immobilized through physical adsorption on the surface of an inorganic nanoparticle of 1 to 500 nm in average particle size.

### TECHNICAL FIELD

The present invention relates to an inorganic nanoparticle for use in fields such as life science or medical diagnosis. More particularly, the present invention relates to an inorganic nanoparticle having a drug immobilized on the surface thereof.

### BACKGROUND ART

Inorganic nanoparticles, except for gold colloid, have an inactive surface and therefore indispensably required a reaction via a compound having a functional group, such as a silane coupling agent, for binding a variety of organic compounds to the surface thereof. Thus, a multistage treatment is required for linking a compound of interest to inorganic nanoparticles. While usual organic compounds are not magnetic, an organic substance can be operated magnetically through a magnetic substance by forming a composite of the magnetic substance with the organic compound. A composite where an organic compound is immobilized on a fine particle of a magnetic substance and can thereby be operated magnetically is applicable in many fields including biology and medical science.

For example, affinity beads for isolating and immobilizing receptors of drugs or chemical substances have been developed and applied. The beads have the potential of expanding their applications to more than drug development by making the beads magnetically responsive. Non-Patent Document 1 has described the synthesis of ferrite magnetic nanoparticles to which functionality as beads for HTS has been imparted, and has also described the possibility of their being developed to an MRI contrast medium, DDS, sensor, and so on as their future applications. Alternatively, Non-Patent Document 2 has described solution, coprecipitation, microemulsion, polyol, high-temperature deposition, and spray pyrolysis methods, and so on as synthetic methods of magnetic nanoparticles. This document has reported hyperthermia and drug delivery as in-vivo therapeutic applications of magnetic nanoparticles and magnetic resonance imaging (MRI) as diagnostic applications thereof. Moreover, the document has described the in-vitro application of magnetic nanoparticles to separation and purification or to Magnetorelaxometry and so on.

As described in Non-Patent Documents 1 and 2 above, organic substances such as selective-binding proteins or cells are bound to magnetic carrier particles, and the proteins or cells are separated and extracted by utilizing their selective binding properties and the magnetic separation; or substances such as drugs are delivered by the action of magnetic force thereon. These documents have also discussed the image sharpening of magnetic resonance imaging (MRI) diagnosis using magnetic nanoparticles and the use of magnetic nanoparticles in the local heating of the affected part by use of their properties of producing heat by electromagnetic waves.

Furthermore, Patent Document 1 has described a composite material of an organic substance compounded with ferrite, which is capable of retaining a stable bond under use conditions and, if necessary, capable of binding. A functional group carrying sulfur atoms such as a mercapto group is held in an organic substance and allowed to act on the surface of ferrite to thereby provide for a stable chemical bond therebetween, thereby forming a composite material of the organic substance and the ferrite. For example, a physiologically active substance or biological substance can be used as the organic substance. The organic substance can be operated magnetically by this compounding. Various physiologically active substances can be bound to the composite material by introducing a second functional group into the organic substance. Specifically, in Patent Document 1, sulfur atoms are used in the binding of an organic compound with ferrite to produce a composite of the organic compound and the ferrite, thereby sensitizing images of magnetic resonance imaging diagnosis. In any case, magnetic particles are modified with polymers, low-molecular linkers, lipid, or the like, and then, a physiologically active compound is immobilized thereon by a chemical bond via these molecules. However, this method presented problems such as poor versatility because the method causes decreases in the amount of a physiologically active compound immobilized or requires the introduction of a particular functional group.

There has so far been no report about a method for directly immobilizing a medicinal ingredient of interest onto magnetic nanoparticles.

[Non-Patent Document 1] Construction of a high functional magnetic nanobeads and its application to biotechnology, BIO INDUSTRY Vol. 21, No. 8, 21-30, 2004, Nobuyuki Gokon, Kosuke Nishio, Hiroshi Handa
[Non-Patent Document 2] The preparation of magnetic nano-particles for applications in biomedicine, J. Phys. D: Apply. Phys. 36 (2003), 182-197, Pedro Tartaj et al

### [Patent Document 1] Japanese Patent Laid-Open No. 2005-60221

### DISCLOSURE OF THE INVENTION

An object to be attained by the present invention is to resolve the problems of the conventional techniques described above. Specifically, an object to be attained by the present invention is to provide a composite of an inorganic nanoparticle with a medicinal compound (i.e., an active substance), and to provide a method for producing the inorganic nanoparticle composite, which is highly versatile and convenient.

The present inventors have conducted diligent studies to attain the object and have consequently completed the present invention by finding that an inorganic nanoparticle holding a drug on the surface thereof can be formed by mixing a solution of a medicinal compound with a dispersion of inorganic nanoparticles and irradiating the resulting mixture solution with ultrasonic waves.

Specifically, the present invention provides an active substance-immobilized inorganic nanoparticle wherein an active substance is immobilized through physical adsorption on the surface of an inorganic nanoparticle of 1 to 500 nm in average particle size.

Preferably, the inorganic nanoparticle is a nanoparticle of a magnetic substance.

Preferably, the inorganic nanoparticle is iron oxide or ferrite.

Preferably, the active substance is immobilized through physical adsorption on the surface of the inorganic nanoparticle comprising an amino acid immobilized on the surface.

Preferably, an amino acid is immobilized on the surface of the inorganic nanoparticle surface-modified with a compound represented by the formula R¹—(OCH(R²)CH₂)ₙ—O-L-X (wherein R¹ represents an alkyl or alkenyl group having a carbon chain length between 1 and 20 inclusive or an unsubstituted phenyl group or phenyl group substituted with an alkyl or alkoxyl group having a carbon chain length of 10 or less; R₂ represents a hydrogen atom or methyl group; n represents an integer between 1 and 20 inclusive; L represents a single bond or alkylene group having 1 to 10 carbon atoms; and X represents a carboxylic acid group, phosphoric acid group, sulfonic acid group, or boric acid group), and the active substance is further immobilized through physical adsorption on the surface.

Another aspect of the present invention provides a method for producing the inorganic nanoparticle of the present invention, which comprises irradiating, with ultrasonic waves, a mixture of an active substance with a dispersion of inorganic nanoparticles of 1 to 500 nm in average particle size.

Preferably, a time of the irradiation with ultrasonic waves is between 1 minute and 2 hours inclusive.

Preferably, the irradiation is performed with ultrasonic waves having a high-frequency output of 0.1 to 200 W.

A further another aspect of the present invention provides an inorganic nanoparticle produced by the method of the present invention described above.

A further another aspect of the present invention provides a hyperthermia agent which comprises the inorganic nanoparticle of the present invention described above.

A further another aspect of the present invention provides an MRI contrast medium which comprises the inorganic nanoparticle of the present invention described above.

A further another aspect of the present invention provides a drug delivery agent which comprises the inorganic nanoparticle of the present invention described above.

A further another aspect of the present invention provides a probe for analysis and diagnosis which comprises the inorganic nanoparticle of the present invention described above.

A further another aspect of the present invention provides a separating agent for a physiologically active substance which comprises the inorganic nanoparticle of the present invention described above.

A further another aspect of the present invention provides a method for magnetically separating and purifying a physiologically active substance, which comprises bringing the inorganic nanoparticle of the present invention into contact with a physiologically active substance.

### BEST MODE FOR CARRYING OUT THE INVENTION

Hereinafter, the embodiments of the present invention will be described in detail.

The present invention relates to an active ingredient-immobilized inorganic nanoparticle, and to a method for producing the inorganic nanoparticle which comprises sonicating a mixture of a water-dispersible nanoparticle with an active ingredient. The active substance-immobilized inorganic nanoparticle according to the present invention is characterized in that an active substance is immobilized through physical adsorption on the surface of an inorganic nanoparticle of 1 to 500 nm in average particle size.

The inorganic nanoparticle used in the present invention includes, but not limited to, iron oxide nanoparticles, zinc oxide nanoparticles, titanium oxide nanoparticles, silica nanoparticles, and alumina nanoparticles. Preferably, the inorganic nanoparticle can include magnetic nanoparticles.

Preferably, independently dispersed magnetic nanoparticles of 1 to 50 nm in average particle size are used as the inorganic nanoparticle of 1 to 500 nm in average particle size of the present invention. The phrase “independently dispersed” means a state in which the particles are individually dispersed in a solution without forming aggregates. Moreover, the average particle size of the magnetic nanoparticles is preferably 1 to 50 nm, more preferably 1 to 40 nm or smaller, particularly preferably 1 to 30 nm or smaller.

Any particle can be used as the magnetic nanoparticle as long as the particle can be dispersed or suspended in an aqueous medium and can be separated from the dispersion or suspension by applying a magnetic field thereto. Examples of the magnetic nanoparticle used in the present invention include: salts, oxides, borides, or sulfides of iron, cobalt, or nickel; and rare earth elements having high magnetic susceptibility (e.g., hematite or ferrite). Additional specific examples of the magnetic nanoparticle that can be used include ferromagnetic ordered alloys such as magnetite (Fe₃O₄), FePd, FePt, and CoPt. In the present invention, a preferable magnetic nanoparticle is selected from the group consisting of metal oxides, particularly, iron oxide and ferrite (Fe,M)₃O₄. In this context, the iron oxide particularly includes magnetite, maghemite, and mixtures thereof. In the formula, M represents a metal ion that can form a magnetic metal oxide together with the iron ion. This metal ion is typically selected among transition metals and is most preferably Zn²⁺, Co²⁺, Mn²⁺, Cu²⁺, Ni²⁺, Mg²⁺, or the like. An M/Fe molar ratio is determined according to the stoichiometric composition of the selected ferrite. The metal salt is supplied in a solid or a solution form and is preferably a chloride salt, bromide salt, or sulfate. Of them, iron oxide and ferrite are preferable from a safety viewpoint. Particularly preferably, the magnetic nanoparticle is magnetite (Fe₃O₄).

A dispersion of inorganic (magnetic) nanoparticles used in the present invention can be prepared, for example, by dispersing inorganic nanoparticle aggregates by the addition of an aqueous solution of a surfactant (e.g., polyoxyethylene (4,5) lauryl ether acetate). However, a preparation method of the dispersion of magnetic nanoparticles is not limited to this method. For example, a hydrophilic polymer [e.g., polyethylene glycol or sodium polyphosphate] or phospholipid (e.g., phosphatidylcholine) may be allowed to coexist during or after nanoparticle synthesis.

Preferably, a compound represented by the following formula can also be used in the present invention:

Formula: R¹—(OCH(R²)CH₂)ₙ—O-L-X

wherein R¹ represents an alkyl or alkenyl group having a carbon chain length between 1 and 20 inclusive or an unsubstituted phenyl group or phenyl group substituted with an alkyl or alkoxyl group having a carbon chain length of 10 or less; R₂ represents a hydrogen atom or methyl group; n represents an integer between 1 and 20 inclusive; L represents a single bond or alkylene group having 1 to 10 carbon atoms; and X represents a carboxylic acid group, phosphoric acid group, sulfonic acid group, or boric acid group.

The alkyl group having a carbon chain length between 1 and 20 inclusive can include a methyl group, ethyl group, n-propyl group, isopropyl group, t-butyl group, octyl group, and cetyl group. The alkenyl group having a carbon chain length between 1 and 20 inclusive can include the alkyl groups described above having at least one or more double bonds.

In the present invention, specific examples of the compound represented by the formula include, but not limited to, the following compounds:

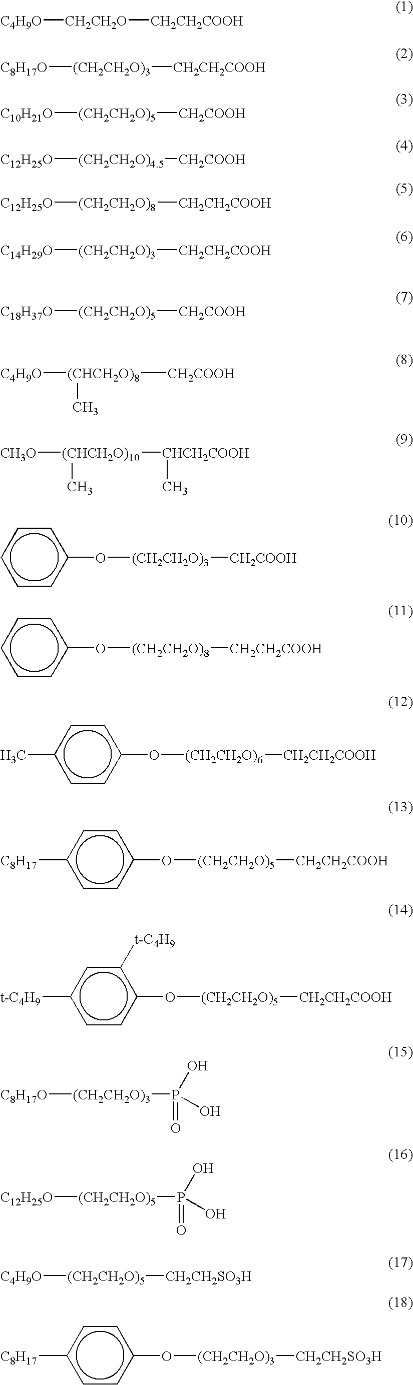

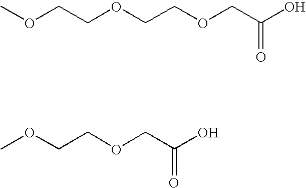

For the inorganic nanoparticle of the present invention, preferably 1 to 200 amino acid molecules, more preferably 1 to 100 amino acid molecules are immobilized per magnetic nanoparticle. The amino acid to be immobilized can include glycine, alanine, valine, leucine, isoleucine, norvaline, norleucine, serine, threonine, aspartic acid, glutamic acid, asparagine, glutamine, lysine, arginine, cysteine, methionine, ornithine, citrulline, phenylalanine, tyrosine, tryptophan, histidine, β-alanine, γ-aminobutyric acid (GABA), and proline. The amino acid to be immobilized is preferably a water-soluble amino acid and can be selected among, for example, glycine, alanine, serine, threonine, aspartic acid, glutamic acid, lysine, arginine, cysteine, proline, β-alanine, and GABA.

The inorganic nanoparticle comprising the amino acid immobilized on the surface thereof can be produced, for example, by a treatment in which the inorganic nanoparticles of 1 to 500 nm in average particle size which is dispersed in water are irradiated with ultrasonic waves in the presence of the amino acid.

In the present invention, the irradiation with ultrasonic waves which is performed for immobilizing the amino acid on the surface of the inorganic nanoparticle can be performed according to a standard method generally known by those skilled in the art and can be performed, for example, by using a commercially available ultrasonic bath. Preferably, the irradiation with ultrasonic waves is performed in a buffer solution at pH 5.0 or higher and can be performed, for example, in a phosphate buffer solution. A time of the irradiation with ultrasonic waves is not particularly limited and can be set appropriately as long as the time allows for the immobilization of the amino acid on the surface of the magnetic nanoparticle. In general, the time is between 1 minute and 2 hours inclusive. Preferably, the irradiation is performed with ultrasonic waves having a high-frequency output of 0.1 to 200 W.

In the inorganic nanoparticle of the present invention, the active substance is immobilized through physical adsorption on the surface of the inorganic nanoparticle. The active substance used in the present invention is a cosmetic ingredient such as moisturizing agent, skin-whitening agent, or anti-aging agent, a functional food ingredient such as a vitamin or antioxidant, or a pharmaceutical ingredient such as an anticancer agent, antiallergic agent, antithrombotic agent, or antiinflammatory agent.

Specific examples of moisturizing agents used in the present invention include, but are not limited to, hyaluronic acid, ceramide, Lipidure, isoflavone, amino acid, and collagen.

Specific examples of skin-whitening agents used in the present invention include, but are not limited to, vitamin C, arbutin, hydroquinone, kojic acid, Lucinol, and ellagic acid.

Specific examples of anti-aging agents used in the present invention include, but are not limited to, retinoic acid, retinol, vitamin C, kinetin, β-carotene, astaxanthin, and tretinoin.

Specific examples of an antioxidant that can be used in the present invention include, but are not limited to, vitamin C derivative, vitamin E, kinetin, α-lipoic acid, and coenzyme Q10.

Specific examples of an anticancer agent that can be used in the present invention include, but are not limited to, pyrimidine fluoride antimetabolites (e.g., 5-fluorouracil (5FU), tegafur, doxifluridine, and capecitabine), antibiotics (e.g., mitomycin (MMC) and Adriacin (DXR)), purine antimetabolites (e.g., folic acid antimetabolites such as methotrexate, and mercaptopurine), vitamin A active metabolites (e.g., antimetabolites such as hydroxy carbamide, tretinoin, and tamibarotene), molecular targeting agents (e.g., Herceptin and imatinib mesylate), platinum drugs (e.g., Briplatin and Randa (CDDP), Paraplatin (CBDC), Elplat (Oxa), and Aqupla), plant alkaloids (e.g., Topotecin, Campto (CPT), Taxol (PTX), Taxotere (DTX), and Etoposide), alkylating agents (e.g., Busulfan, cyclophosphamide, and Ifomide), antiandrogens (e.g., bicalutamide and flutamide), female hormones (e.g., Fosfestrol, chlormadinone acetate, and estramustine phosphate), LH-RH agonists (e.g., Leuplin and Zoladex), antiestrogens (e.g., tamoxifen citrate and toremifene citrate), aromatase inhibitors (e.g., fadrozole hydrochloride, anastrozole, and Exemestane), progestins (e.g., medroxyprogesterone acetate), and BCG.

Specific examples of antiallergic agents used in the present invention include, but are not limited to: mediator antireleasers, such as disodium cromoglycate and tranilast; histamine H1 antagonists, such as ketotifen fumarate and azelastine hydrochloride; thromboxane inhibitors, such as ozagrel hydrochloride; leukotriene antagonists, such as pranlukast; and suplatast tosylate.

The active substance used in the present invention may be used alone or in combination of two or more types.

In the present invention, the irradiation with ultrasonic waves which is performed for immobilizing the active substance on the surface of the inorganic nanoparticle can be performed according to a standard method generally known by those skilled in the art and can be performed, for example, by using a commercially available ultrasonic bath. The irradiation with ultrasonic waves can be performed, for example, in water. A time of the irradiation with ultrasonic waves is not particularly limited and can be set appropriately as long as the time allows for the immobilization of the drug on the surface of the nanoparticle. In general, the time is between 1 minute and 2 hours inclusive. Preferably, the irradiation is performed with ultrasonic waves having a high-frequency output of 0.1 to 200 W.

When the inorganic nanoparticle of the present invention is a magnetic nanoparticle, the nanoparticle is magnetic and therefore, can be guided to a predetermined site by magnetic force. Specifically, the magnetic nanoparticle of the present invention can be administered into a living body and guided to the affected part by magnetic force. Moreover, the magnetic nanoparticle thus guided to the affected part can be confirmed by MRI. Specifically, the magnetic nanoparticle of the present invention is useful as a contrast medium for MRI.

The nanoparticle of the present invention comprises an active substance. Such a nanoparticle of the present invention is guided to the affected part according to the method described above and then heated by exposure to ultrasonic waves. As a result, the pharmaceutically active substance incorporated in the nanoparticle can be released. Specifically, the nanoparticle of the present invention is useful as a hyperthermia agent or drug delivery agent.

The nanoparticle of the present invention can further be used as a probe for analysis and diagnosis. Specifically, the nanoparticle can be used in the detection, analysis, condensation, and purification of a variety of amino acid receptors (e.g., glutamic acid receptors, aspartic acid receptors, and serine receptors).

An administration method of the inorganic nanoparticle of the present invention is not particularly limited. Preferably, the inorganic nanoparticle is administered by injection into blood vessels, body cavities, or lymph nodes, particularly preferably by intravenous injection.

The dose of the inorganic nanoparticle of the present invention can be set appropriately according to the body weight of a patient, the state of the disease, and so on. In general, approximately 10 μg to 100 mg/kg can be administered per administration. Preferably, approximately 20 μg to 50 mg/kg can be administered per administration.

When the inorganic nanoparticle of the present invention is a magnetic nanoparticle, the nanoparticle can be used for separating a physiologically active substance in a sample by bringing the nanoparticle into contact with the sample. Specifically, the magnetic nanoparticle of the present invention can be used as a separating agent for a physiologically active substance. Depending on the type of a biological sample, the biological sample can be brought into contact with the magnetic nanoparticle in the presence of an aggregation promoter. In this context, the aggregation promoter is a substance that induces aggregation. Appropriate substances can be used alone or in combination according to the types of fractions to be aggregated.

The present invention will be described more specifically with reference to Examples below. However, the present invention is not limited thereto.

### EXAMPLES

### Production Example 1

### Preparation of Dispersion of Magnetic Nanoparticles

10.8 g of iron (III) chloride hexahydrate and 6.4 g of iron (II) chloride tetrahydrate were separately dissolved in 80 ml of 1 N hydrochloric acid aqueous solution and then mixed together. 96 ml of ammonia water (28% by weight) was added at a speed of 2 ml/min. into this solution with stirring. Subsequently, the resulting solution was heated at 80° C. for 30 minutes and then cooled to room temperature. The obtained aggregate was purified with water by decantation. The generation of magnetite (Fe₃O₄) of approximately 12 nm in crystallite size was confirmed by X-ray diffraction.

This aggregate was dispersed by the addition of 100 ml of an aqueous solution containing 2.3 g of polyoxyethylene (4,5) lauryl ether acetate (Nikko Chemicals Co., Ltd.) dissolved therein (which was adjusted with NaOH to pH 6.8) to prepare a dispersion of magnetically responsive nanoparticles.

### Production Example 2

### Surface Modification of Magnetically Responsive Nanoparticles with Aspartic Acid

1.0 ml of 0.1 M phosphate buffer solution (pH 7.6) and 100 μl of 1 M aspartic acid solution were added to 1.0 ml of the dispersion of magnetically responsive nanoparticles (iron oxide content of 18.2 g/L) produced in Production Example 1, which were dispersed in water with the surfactant (polyoxyethylene (4,5) lauryl ether acetate). The resulting mixture solution was irradiated with ultrasonic waves at 100 W for 20 minutes in an ultrasonic bath Sharp UT-105. The aggregated magnetic substances were gathered with a magnet, and the supernatant was removed. After the addition of 2.0 ml of ethanol, the aggregates were washed with a vortex mixer and gathered again with a magnet. The washing solution was discarded. Next, after the addition of 2.0 ml of water, the aggregates were washed with a vortex mixer and gathered again with a magnet. The washing solution was discarded. Finally, after the addition of 2.0 ml of water, the aggregates were irradiated with ultrasonic waves at 100 W for 20 minutes. As a result, the magnetic nanoparticles were uniformly redispersed and prepared into a colorless dispersion. When the zeta potential of the magnetic nanoparticles was measured, it was changed to −24 mV from −31 mV measured before the treatment, demonstrating that the surfaces were substituted by aspartic acid.

### Example 1

### Surface Modification of Magnetically Responsive Nanoparticles with Adriamycin

1.0 ml of the dispersion of the aspartic acid-modified magnetic nanoparticles (Fe₃O₄ content of 1.0 mg/ml) and an Adriamycin aqueous solution (1.0 mg/ml) were mixed and irradiated with ultrasonic waves at 100 W for 20 minutes by use of an ultrasonic bath Sharp UT-105. The aggregated magnetic substances were gathered with a magnet, and the supernatant was separated. The amount of Adriamycin remaining (Abs. 480 nm) was measured from the absorption spectrum of the supernatant to calculate the amount of Adriamycin immobilized on the surfaces of the magnetic substances. The magnetic nanoparticle aggregates separated with a magnet were redispersed with a vortex mixer after the addition of 1.0 ml of water.

The amount of Adriamycin immobilized was 200 μg/1.0 mg of Fe₃O₄. Moreover, the Zeta potential was changed to +17.7 mV from −24 mV, indicating that the amino group of Adriamycin was present on the surfaces of the magnetic substances.

### Example 2

### Surface Modification of Magnetically Responsive Nanoparticles with Astaxanthin

1.0 ml of the magnetic nanoparticles (Fe₃O₄ content of 1.0 mg/ml) produced in Production Example 1, which were dispersed in water with the surfactant (polyoxyethylene (4,5) lauryl ether acetate), and 1.0 ml of 100 ppm astaxanthin/1% ascorbic acid aqueous solution were mixed and irradiated with ultrasonic waves at 100 W for 20 minutes by use of an ultrasonic bath Sharp UT-105. The aggregated magnetic substances were gathered with a magnet, and the supernatant was separated. As a result, the supernatant became colorless, demonstrating that astaxanthin in the solution was quantitatively immobilized on the magnetically responsive nanoparticles.

### INDUSTRIAL APPLICABILITY

The present invention has made it possible to provide a versatile and convenient method capable of producing a drug-immobilized inorganic nanoparticle.

1. An active substance-immobilized inorganic nanoparticle wherein an active substance is immobilized through physical adsorption on the surface of an inorganic nanoparticle of 1 to 500 nm in average particle size.

2. The inorganic nanoparticle of claim 1 wherein the inorganic nanoparticle is a nanoparticle of a magnetic substance.

3. The inorganic nanoparticle of claim 1 wherein the inorganic nanoparticle is iron oxide or ferrite.

4. The inorganic nanoparticle of claim 1 wherein the active substance is immobilized through physical adsorption on the surface of the inorganic nanoparticle comprising an amino acid immobilized on the surface.

5. The inorganic nanoparticle of claim 1 wherein an amino acid is immobilized on the surface of the inorganic nanoparticle surface-modified with a compound represented by the formula R¹—(OCH(R²)CH₂)ₙ—O-L-X
wherein R¹ represents an alkyl or alkenyl group having a carbon chain length between 1 and 20 inclusive or an unsubstituted phenyl group or a phenyl group substituted with an alkyl or alkoxyl group having a carbon chain length of 10 or less; R² represents a hydrogen atom or methyl group; n represents an integer between 1 and 20 inclusive; L represents a single bond or an alkylene group having 1 to 10 carbon atoms; and X represents a carboxylic acid group, a phosphoric acid group, a sulfonic acid group, a or boric acid group,
and the active substance is further immobilized through physical adsorption on the surface.

6. A method for producing the inorganic nanoparticle of claim 1, which comprises irradiating, with ultrasonic waves, a mixture of an active substance with a dispersion of inorganic nanoparticles of 1 to 500 nm in average particle size.

7. The method of claim 6 wherein the time of the irradiation with ultrasonic waves is between 1 minute and 2 hours inclusive.

8. The method of claim 6 wherein the irradiation is performed with ultrasonic waves having a high-frequency output of 0.1 to 200 W.

9. An inorganic nanoparticle produced by the method of claim 6.

10. A hyperthermia agent which comprises the inorganic nanoparticle of claim 1.

11. An MRI contrast medium which comprises the inorganic nanoparticle of claim 1.

12. A drug delivery agent which comprises the inorganic nanoparticle of claim 1.

13. A probe for analysis and diagnosis which comprises the inorganic nanoparticle of claim 1.

14. A separating agent for a physiologically active substance which comprises the inorganic nanoparticle of claim 1.

15. A method for magnetically separating and purifying a physiologically active substance, which comprises bringing the inorganic nanoparticle of the present invention into contact with a physiologically active substance.

16. A hyperthermia agent which comprises the inorganic nanoparticle of claim 9.

17. An MRI contrast medium which comprises the inorganic nanoparticle of claim 9.

18. A drug delivery agent which comprises the inorganic nanoparticle of claim 9.

19. A probe for analysis and diagnosis which comprises the inorganic nanoparticle of claim 9.

20. A separating agent for a physiologically active substance which comprises the inorganic nanoparticle of claim 9.
